(19) United States
(12) Patent Application Publication
(10) Pub. No.: US 2008/0286234 A1
(43) Pub. Date: Nov. 20, 2008

(54) Method for treating demyelinating diseases

(76) Inventor: Daniel A. Eyink, Newburyport, MA (US)

Correspondence Address: Paul G. Lunn, 14606 - 135th Ct. N.E., Woodinville, WA, 98072-4607, US

(21) Appl. No.: 12/152,396

(22) Filed: May 14, 2008

Related U.S. Application Data
(60) Provisional application No. 60/930,272, filed on May 15, 2007.

(51) Int. Cl. A61K 38/21; A61K 31/519; A61P 25/00; A61K 31/59

(52) U.S. Cl. 424/85.6; 514/259.41; 514/167

(57) ABSTRACT

A method for treating demyelinating diseases such as MS comprised of administering to an individual afflicted with a demyelinating disease a medicament that induces prolactin secretion. Prolactin secretion stimulates oligodendrocyte precurser cells, which remyelinate demyelinated nerve cells. The medicament is administered at a dosage sufficient to induce prolactin secretion resulting in a hyperprolactinemia. Estrogen, estradiol, estriol, histamine H₂-receptor antagonists such as cimetidine, and vitamin D can be administered in conjunction with the prolactin-inducing medicament to further promote the remyelination of the nerve cells.

This claims benefit of U.S. Provisional Application Ser. No. 60/930,272 filed on May 15, 2007 the contents of which are incorporated in their entirety by reference.

### BACKGROUND OF THE INVENTION

Myelin sheaths, cover many nerve fibers, are composed of lipoprotein layers formed in early life. Myelin formed by the oligodendroglia in the central nervous system (CNS) differs chemically and immunologically from that formed by the Schwann cells peripherally, but both types have the same function to promote transmission of a neural impulse along an axon.

Demyelination in later life is a feature of many neurologic disorders. It can result from damage to nerves or myelin due to local injury, ischemia, toxic agents, or metabolic disorders. Extensive myelin loss is usually followed by axonal degeneration and often by cell body degeneration, both of which may be irreversible.

Multiple Sclerosis (abbreviated MS, and the closely related condition disseminated sclerosis or encephalomyelitis disseminata are chronic, inflammatory diseases that affect the central nervous system (CNS). MS can cause a variety of symptoms, including changes in sensation, visual problems, muscle weakness, depression, difficulties with coordination and speech, severe fatigue, cognitive impairment, problems with balance, overheating, and pain. MS will cause impaired mobility and disability in more severe cases.

The following is a list of demyelinating diseases.

Acute disseminated encephalomyelitis is characterized by perivascular CNS demyelination, which can occur spontaneously but usually follows a viral infection (or very rarely, bacterial vaccination), suggesting an immunologic cause.

Adrenoleukodystrophy/Adrenomyeloneuropathy are X-linked recessive metabolic disorders characterized by adrenal gland dysfunction and widespread demyelination of the nervous system. Adrenoleukodystrophy occurs in young boys, and adrenomyeloneuropathy in adolescents. Mental deterioration, spasticity, and blindness may occur.

Leber's Hereditary Optic Atrophy and related mitochondrial disorders are characterized primarily by bilateral loss of central vision, usually affecting males in their late teens or early twenties. Leber's hereditary optic atrophy can resemble the optic neuritis in MS.

HTLV-associated Myelopathy is a slowly progressive spinal cord disease associated with infection by the human T-cell lymphotrophic virus, and is characterized by spastic weakness of both legs.

Multiple Sclerosis (MS) is a slowly progressive CNS disease characterized by disseminated patches of demyelination in the brain and spinal cord, resulting in multiple and varied neurologic symptoms and signs, usually with remissions and exacerbations. MS affects neurons, the cells of the brain and spinal cord that carry information, create thought and perception, and allow the brain to control the body. Surrounding and protecting some of these neurons is a fatty layer known as the myelin sheath, which helps neurons carry electrical signals. MS causes gradual destruction of myelin (demyelination) and transection of neuron axons in patches throughout the brain and spinal cord. The name multiple sclerosis refers to the multiple scars (or scleroses) on the myelin sheaths. This scarring causes symptoms, which vary widely depending upon which signals are interrupted.

The predominant theory today is that MS results from attacks by an individual's immune system on the nervous system and it is therefore usually categorized as an autoimmune disease. There is a minority view that MS is not an autoimmune disease, but rather a metabolically dependent neurodegenerative disease. Although much is known about how MS causes damage, its exact cause remains unknown.

Multiple sclerosis may take several different forms, with new symptoms occurring either in discrete attacks or slowly accruing over time. Between attacks, symptoms may resolve completely, but permanent neurologic problems often persist, especially as the disease advances. MS currently does not have a cure, though several treatments are available which may slow the appearance of new symptoms. MS primarily affects adults, with an age of onset typically between 20 and 40 years, and is more common in women than in men.

The present treatments have been limited to immunomodulatory agents administered to alter the immune system to halt progression of the disease. Examples of these agents are interferon beta-1a, interferon beta-1b, which are drugs in the interferon family used to treat multiple sclerosis (MS). The beta interferons have been shown to have about a 30-35% reduction in the rate of MS relapses, and to slow the progression of disability in MS patients. It is believed that the Interferon beta drugs achieve their beneficial effect on MS progression via their anti-inflammatory properties. Studies have also determined that Interferon beta improves the integrity of the blood-brain barrier (BBB), which generally breaks down in MS patients, allowing increasing amounts of undesirable substances to reach the brain. This strengthening of the BBB may be a contributing factor to Interferon-Beta's beneficial effects.

Nonetheless, Interferons have side effects. The two main ones are flu-like symptoms, and injection-site reactions. The flu-like symptoms tend to happen immediately after the injection, and last for about half a day. In many patients, these symptoms diminish over time, but some patients continue to experience them over the long term. One can mitigate these symptoms by using a dose that is injected less frequently, and by taking the medication before bedtime. Side effects are often onerous enough that many patients ultimately discontinue taking Interferons (or Copaxone®, one of the other disease-modifying therapies requiring daily or weekly injections).

The most commonly reported side effects are injection site disorders, flu-like symptoms, elevation of liver enzymes and blood cell abnormalities.

While these drugs improve certain diagnostic test results, many patients report no perceived improvement, along with serious side effects that substantially reduce quality of life. It is important to recognize that these drugs are intended to treat symptoms and possibly delay disease progression, but do not provide a cure to multiple sclerosis. Furthermore, tolerance develops over time in some patients, due to the development of “neutralizing antibodies,” which reduces the effectiveness of these drugs, while side effects may persist even after discontinuation.

Another drug used to treat MS is glatiramer acetate, which is a synthetic medication peptide made of four amino acids that are found in myelin. This drug stimulates T cells in the body's immune system to change from harmful, pro-inflammatory agents to beneficial, anti-inflammatory agents that work to reduce inflammation at lesion sites. It is also possible that peptide acts as a sort of decoy, thus allowing myelin to regenerate. Glatiramer acetate has been shown in clinical trials to reduce the number and severity of exacerbations and reduce the number of new, gadolinium-enhanced brain lesions on MRI scans.

Another drug used to treat MS is Mitoxantrone, which is an anthracycline antineoplastic agent used in the treatment of certain types of cancer, mostly metastatic breast cancer, acute myeloid leukemia, and non-Hodgkin's lymphoma.

Mitoxantrone is also used to treat multiple sclerosis (MS), most notably the subset known as secondary progressive MS. Mitoxantrone will not cure multiple sclerosis, but is effective in slowing the progression of secondary progressive MS and extending the time between relapses in relapsing-remitting MS and progressive relapsing MS. Mitoxantrone is a type II topoisomerase inhibitor; it disrupts DNA synthesis and DNA repair in both healthy cells and cancer cells.

As other drugs in its class, mitoxantrone may cause several adverse reactions of varying severity, such as nausea, vomiting, hair loss, heart damage, and immunosuppression. Some side effects may have delayed onset. Cardiomyopathy is a particularly concerning effect as it is irreversible; regular monitoring with echocardiograms or MUGA scans are recommended for people taking mitoxantrone.

Another drug used to treat MS Natalizumab is a monoclonal antibody against integrin-(4 that has proven efficacy in the treatment of two serious autoimmune disorders: multiple sclerosis (MS) and Crohn's disease (CD). In MS, natalizumab was shown to reduce relapses by 67% vs. a placebo. It slowed the progression of disability (as measured by EDSS) by 42%. While it is impossible to compare results across different clinical trials, the older generation drugs, interferons and glatiramer acetate (Copaxone), are generally acknowledged as demonstrating about a 30-35% decrease in relapse rate vs. placebo; and only two drugs have been shown to decrease the progression of disability, but again only by around 20-40%. The mechanism of action of natalizumab is believed to involve the inhibition of immune cells from crossing blood vessel walls to reach various tissues, including the brain.

Relapsing-remitting symptomatic attacks can be treated. Patients are typically given high doses of intravenous corticosteroids, such as methylprednisolone, to end the attack sooner and leave fewer lasting deficits. Patients' self-reporting indicates that many find benefit from a number of other medicines. Currently there are no approved treatments for primary progressive multiple sclerosis, though several medications are being studied.

The prognosis (expected future course of the disease) for a person with multiple sclerosis depends on the subtype of the disease; the individual's sex, race, age, and initial symptoms; and the degree of disability the person experiences. The life expectancy of people with MS is now nearly the same as that of unaffected people. This is due mainly to improved methods of limiting disability, such as physical therapy and speech therapy, and more successful treatment of common complications of disability, such as pneumonia and urinary tract infections.

Individuals with progressive subtypes of MS, particularly the primary progressive subtype, have a more rapid decline in function. In the primary progressive subtype, supportive equipment (such as a wheelchair or standing frame) is often needed after six to seven years. However, when the initial disease course is the relapsing-remitting subtype, the average time until such equipment is needed is twenty years. This means that many individuals with MS will never need a wheelchair.

The earlier in life MS occurs, the slower disability progresses. Individuals who are older than fifty when diagnosed are more likely to experience a chronic progressive course, with more rapid progression of disability. Those diagnosed before age 35 have the best prognosis. Females generally have a better prognosis than males. Although black individuals tend to develop MS less frequently, they are often older at the time of onset and may have a worse prognosis.

Initial MS symptoms of visual loss or sensory problems, such as numbness or tingling, are markers for a relatively good prognosis, whereas difficulty walking and weakness are markers for a relatively poor prognosis. Better outcomes are also associated with the presence of only a single symptom at onset, the rapid development of initial symptoms, and the rapid regression of initial symptoms.

The degree of disability varies among individuals with MS. In general, one of three individuals will still be able to work after 15-20 years. Fifteen percent of people diagnosed with MS never have a second relapse, and these people have minimal or no disability after ten years. The degree of disability after five years correlates well with the degree of disability after fifteen years. This means that two-thirds of people with MS with low disability after five years will not get much worse during the next ten years. It should be noted that most of these outcomes were observed before the use of medications such as interferon, which can delay disease progression for several years.

Thus, the drugs currently on the market merely inhibit the progression of MS but do very little in promoting healing of the myelin sheaths surrounding the axons of the neurons. Thus, there is a need for therapeutic agents that actively promote the regeneration of myelin sheath surrounding neuronal axons.

### DESCRIPTION OF THE INVENTION

The present invention fills this need by providing a method for treating demyelinating diseases such as MS comprised of administering to an individual afflicted with a demyelinating disease a medicament that induces prolactin secretion. Prolactin secretion stimulates oligodendrocyte precurser cells, which remyelinate demyelinated nerve cells. The medicament is administered at a dosage sufficient to induce prolactin secretion resulting in a hyperprolactinemia. Prolactin secretion in the pituitary is normally suppressed by the brain chemical, dopamine. Drugs that block the effects of dopamine at the pituitary or deplete dopamine stores in the brain cause the pituitary to secrete prolactin.

These drugs include the major tranquilizers (phenothiazines), trifluoperazine (15-30 mg/day) (Stelazine), histamine H₂-receptor antagonists such as cimetidine (600-1200 mg/day), ranitidine (150 mg-450 mg/day), famotidine 40 mg-80 mg/day), and nizatidine 300 mg-600 mg/day). Also, estrogens and thyrotropin-releasing hormone (TRH) can be used. Examples of agents are the dopamine D₂ receptor antagonists such as the 1,2-benzisothiazoles an example of which is risperidone. Other medicaments include but are not limited to cis-thiothixene (20 mg-30 mg/day), sertindole, fluphenazine (20 mg-30 mg/day), zotepine, perphenazine (60-100 mg/day), thioridazine (800 mg/day), pimozide, haloperidol (15-45 mg/day), ziprasidone, mesoridazine (400 mg/day), sulpiride, olanzapine (20-45 mg/day), chlorpromazine (500 mg-1000 mg/day), loxapine (100-250 mg/day), pipamperone, molindone (150-225 mg/day), amperozide, quetiapine, clozapine (900 mg/day), melperone, and remoxipride.

Hyperprolactinemia is the presence of abnormally high levels of prolactin in the blood. Normal levels are less than 580 mIU/L for women, and less than 450 mIU/L for men. The hormone prolactin is down regulated by dopamine and is up regulated by estrogen.

The treatment of demyelinating diseases according to the present invention will be exemplified by the following description of the use of agents that induce secretion of prolactin to treat MS.

Diagnosis

The diagnosis of MS is indirect and requires documentation of two or more episodes of symptoms and two or more signs that reflect pathology in anatomically noncontiguous white matter tracts of the CNS. Symptoms must last more than one day and occur as distinct episodes that are separated by 28 or more days. At least one of the two required signs must be present on neurological examination. The second may be documented as an abnormal test, either brain or spinal cord MRI, or visual, auditory, or somatosensory evoked electrical response. In patients who experience gradual progression of disability for six month without superimposed relapses, documentation of intrathecal IgG may be used to support the diagnosis.

Examples of Diagnostic Criteria for MS are the following.

1. Examination must reveal objective abnormalities of the CNS.

2. Involvement must reflect predominantly disease of white matter long tracts, usually including (a) pyramidal pathways, (b) cerebellar pathways, (c) medial longitudinal fasciculus, (d) optic nerve, and (e) posterior columns.

3. Examination or history must implicate involvement of two or more areas of the CNS.
(a) MRI may be used to document a second lesion when only one site of abnormality has been demonstrable on examination. A confirmatory MRI must have either four lesions involving the white matter or three lesions, if one is periventricular in location. Acceptable lesions must be >3 mm in diameter. For patients older than 50 years, two of the criteria must be met: (1) lesion size >5 mm, (2) lesions adjacent to the bodies of the lateral ventricles, and (3) one or more lesions present in the posterior fossa.
(b) Evoked response testing may be used to document a second lesion not evident on clinical examination.

4. The clinical patter must consist of (a) two or more separate episodes of worsening involving different sites of the CNS, each lasting least 24 hours and occurring at least 1 month apart or (b) gradual or stepwise progression over at least 6 months, if accompanied by increased cerebral spinal fluid (CSF) IgG synthesis or two or more oligoclonal bands.

5. Age of onset between 15 and 60 years.

6. The patient's neurologic condition could not better be attributed to another disease. Laboratory testing that may be advisable in certain cases includes (a) CSF analysis, (b) MRI of the head or spine, (c) serum vitamin B12 level, (d) human T cell lymphotropic virus type 1 (HTLV-1) titer, (e) erythrocyte sedimentation rate, (f) rheumatoid factor, antinuclear, anti-DNA antibodies (SLE), (g) serum VDRL, (h) angiotensin-converting enzyme (sarcoidosis), (i) Borrelia serology (Lyme disease), (j) very-long-chain fatty acids (adrenoleukodystrophy) and (k) serum or CSF lactate, muscle biopsy, or mitochondrial DNA analysis (mitrochondrial disorders). If all six criteria are fulfilled, the patient definitely has MS.

If all six criteria are fulfilled, except (a) only one objective abnormality despite two symptomatic episodes or (b) only one symptomatic episode despite two or more objective abnormalities, then the patient probably has MS.

If all six criteria are fulfilled except only one symptomatic episode and one objective abnormality, the patient is at risk for MS.

Treatment

As was stated above, according to the present invention, a mammal, preferably a human having a demyelinating disease is administered a medicament that induces prolactin secretion. The medicament is administered at a dosage sufficient to induce prolactin secretion resulting in a hyperprolactinemia. Prolactin secretion in the pituitary is normally suppressed by the brain chemical, dopamine. Drugs that block the effects of dopamine at the pituitary or deplete dopamine stores in the brain may cause the pituitary to secrete prolactin.

These drugs include the major tranquilizers (phenothiazines), trifluoperazine (Stelazine), Drugs that block the effects of dopamine at the pituitary or deplete dopamine stores in the brain cause the pituitary to secrete prolactin. These drugs include the major tranquilizers (phenothiazines), trifluoperazine (15-30 mg/day) (Stelazine), histamine H₂-receptor antagonists such as cimetidine (600-1200 mg/day), ranitidine (150 mg-450 mg/day), famotidine 40 mg-80 mg/day), and nizatidine 300 mg-600 mg/day). Also, estrogens and thyrotropin-releasing hormone (TRH) can be used. Examples of agents are the dopamine D₂ receptor antagonists such as the 1,2-benzisothiazoles an example of which is risperidone. Other medicaments include but are not limited to cis-thiothixene (20 mg-30 mg/day), sertindole, fluphenazine (20 mg-30 mg/day), zotepine, perphenazine (60-100 mg/day), thioridazine (800 mg/day), pimozide, haloperidol (15-45 mg/day), ziprasidone, metocloproamide (20 mg-100 mg/day) mesoridazine (400 mg/day), sulpiride, olanzapine (20-45 mg/day), chlorpromazine (500 mg-1000 mg/day), loxapine (100-250 mg/day), pipamperone, molindone (150-225 mg/day), amperozide, quetiapine, clozapine (900 mg/day), melperone, and remoxipride. These drugs may be administered alone or in combination with each other.

A medicament that induces prolactin is administered at a dose high enough to induce prolactin secretion. The secreted prolactin then induces remyelination of the axons of the affected neurons. By way of example, risperidone (RISPERDAL®, marketed by Janssen Pharmaceutica Products, L.P., Titusville, N.J.) is administered at 1 mg to 16 mg or more per day. The drug is administered at a dosage to raise the serum levels of prolactin above normal serum levels, preferably at least 5 to 100% or more above the naturally occurring serum levels in a mammal, specifically a human afflicted with a demyelinating disease such as MS.

The administration of prolactin-inducing drugs can be administered in conjunction with other medicaments such as vitamin D, 17β-estradiol for women, 17α-estradiol for men, estridol, insulin-like growth factor and a serotonin and norepinephrine reuptake inhibitor such as duloxetine HCl (CYMBALTA, Eli Lilly, Indianapolis, Ind.). 30-120 mg.

Preferably a vitamin D such as cholecalciferol (vitamin D3), ergocalciferol (vitamin D2), calcifidiol (25-hydroxycholecalciferol) and calcitriol (1,25-dihydroxycholecalciferol) should be administered in conjunction with the prolactin-inducing agent. For calcitriol the dose should be 0.25 μg to about 2.5 μg without calcium.

If resperidone is administered, it should be administered at a dose of 1-16 mg per day, preferably 8 mg per day (4 mg twice a day) for at least 3 months.

In a preferred embodiment estriol should administered in amount sufficient to raise blood levels of estriol to reach levels comparable to those 3^{rd} trimester pregnancy levels for at least 3 months. Preferably estriol should be administered at a dose of at least 8 mg per day. Estrogen or estradiol can also be administered at 1.5 mg per day, but estriol is preferred due to the lower toxicities of estriol.

A vitamin D, preferably calcitriol, should be administered at a dose from 0.25 μg to about 2.5 μg without calcium. In a preferred embodiment, the amount of calcium ingested by the patient should be 800 mg or less per day for a 3-month treatment period or permanently 800 mg or less.

In a further preferred embodiment, cimetidine is also administered at 600-1200 mg three times per day.

In addition to the above, the individual having MS can be administered orally myelin basic protein in an amount sufficient to induce tolerance and inhibit the demyelination process. See Eylar, E. H. et al. J Biol Chem. 246:5770-5784 (1971)

What is claimed is:

1. A method for treating a demyelinating disease comprising administering to a mammal afflicted with said demyelinating disease a medicament that induces secretion of prolactin in said mammal at a dose high enough to induce hyperprolactinemia.

2. The method of claim 1 wherein the medicament is selected from the group consisting of histamine H₂-receptor antagonists, cimetidine, ranitidine, famotidine, nizatidine phenothiazines, trifluoperazine, haloperidol, metoclopramide, reserpine, estrogens, thyrotropin-releasing hormone, dopamine D₂ receptor antagonists, a serotonin-reuptake inhibitor, 1,2-benzisothiazoles, risperidone, cis-thiothixene sertindole, fluphenazine, zotepine, perphenazine, thioridazine, pimozide, haloperidol, ziprasidone, mesoridazine, sulpiride, olanzapine, chlorpromazine, loxapine, pipamperone, molindone, amperozide, quetiapine, clozapine, melperone, and remoxipride alone or in combination.

3. The method of claim 1 wherein the prolactin-secretion inducing medicament is administered in conjunction with vitamin D or calcitriol.

4. The method of claim 1 wherein the prolactin-secretion inducing medicament is administered in conjunction with a drug selected from the group consisting of natalizumab, a steroid, glatiramer acetate, a beta interferon, myelin basic protein and mitoxantrone.

5. The method of claim 1 wherein the demyelinating disease is a disease selected from the group consisting of acute disseminated encephalomyelitis, adrenoleukodystrophy, adrenomyeloneuropathy, Leber's Hereditary Optic Atrophy, HTLV-associated myelopathy and multiple sclerosis (MS).

6. The method of claim 5 treating wherein the demyelinating disease is MS.

7. The method of claim 1 wherein mammal is a human.

8. A method for treating MS in a mammal comprising administering to said mammal a medicament that induces secretion of prolactin in said mammal at a dose high enough to induce hyperprolactinemia.

9. The method of claim 8 wherein the medicament is selected from the group consisting of histamine H₂-receptor antagonists, cimetidine, ranitidine, famotidine, nizatidine phenothiazines, trifluoperazine, haloperidol, metoclopramide, reserpine, estrogens, thyrotropin-releasing hormone, dopamine D₂ receptor antagonists, a serotonin-reuptake inhibitor, 1,2-benzisothiazoles, risperidone, cis-thiothixene sertindole, fluphenazine, zotepine, perphenazine, thioridazine, pimozide, haloperidol, ziprasidone, mesoridazine, sulpiride, olanzapine, chlorpromazine, loxapine, pipamperone, molindone, amperozide, quetiapine, clozapine, melperone, and remoxipride alone or in combination.

10. The method of claim 9 wherein the medicament is risperidone.

11. The method of claim 10 wherein the risperidone is administered to said mammal at a dose of at least 4 mg per day.

12. The method of claim 11 wherein the risperidone is administered at a dose at least 16 mg per day.

13. The method of claim 8 wherein the prolactin-secretion inducing medicament is administered in conjunction with a vitamin D.

14. The method of claim 8 wherein the prolactin-secretion inducing medicament is administered in conjunction with a drug selected from the group consisting of natalizumab, a steroid, glatiramer acetate, a beta interferon and mitoxantrone.

15. The method of claim 8 wherein the mammal is a human.

16. A method for treating multiple sclerosis (MS) comprising administering to a mammal afflicted with MS risperidone at a dose high enough to induce hyperprolactinemia.

17. The method of claim 16 further comprising administering to said mammal estrogen, estradiol and estriol.

18. The method of claim 16 further comprising administering cimetidine to said mammal.

19. The method of claim 16 further comprising administering a vitamin D to said individual.
